(19) United States
(12) Patent Application Publication
(10) Pub. No.: US 2017/0296175 A1
(43) Pub. Date: Oct. 19, 2017

(54) MEDICAL STAPLE

(71) Applicant: OLYMPUS CORPORATION, Tokyo (JP)

(72) Inventors: Yoshiyuki KUMADA, Tokyo (JP); Kayuri KIMURA, Saitama (JP); Hiroshi OKABE, Tokyo (JP)

(73) Assignee: OLYMPUS CORPORATION, Tokyo (JP)

(21) Appl. No.: 15/588,046

(22) Filed: May 5, 2017

Related U.S. Application Data
(63) Continuation of application No. PCT/JP2014/079732, filed on Nov. 10, 2014.

(51) Int. Cl. A61B 17/068; A61B 17/064; A61B 17/072

(52) U.S. Cl. CPC A61B 17/068; A61B 17/0682; A61B 17/0644; A61B 2017/07264; A61B 2017/07221

(57) ABSTRACT

A medical staple is provided with: a rectangular base portion; and a needle portion that extends from one end of the base portion in a substantially arc shape so as to protrude in a predetermined direction with respect to the base portion, wherein the needle portion has a first bending position that is provided between a base connected at the one end and a distal end, and that yields first when a pressing force in a radially inward direction of an arc shape of the needle portion acts on the distal end, and a second bending position that is provided between the first bending position and the base, and that yields first when the pressing force acts in the vicinity of the base of the first bending position.

### CROSS-REFERENCE TO RELATED APPLICATIONS

This is a continuation of International Application PCT/JP2014/079732, with an international filing date of Nov. 10, 2014, which is hereby incorporated by reference herein in its entirety. This application claims the benefit of International Application PCT/JP2014/079732.

### TECHNICAL FIELD

The present invention relates to a medical staple.

### BACKGROUND ART

In the related art, a stapler has been used to suture pieces of biological tissue together (for example, see Patent Literatures 1 and 2). Staples to be employed as needles for the stapler exist in various shapes, so that it is possible to select ones that are suitable for the shape of biological tissue to be sutured, and so that it is possible to stably cause bending deformation in the staples to bend them into desired shapes. For example, Patent Literature 1 discloses a general U-shaped staple, whereas Patent Literature 2 discloses a C-shaped staple.

### CITATION LIST

### Patent Literature

### {PTL 1} Japanese Unexamined Patent Application, Publication No. 2008-093480

{PTL 2} Publication of U.S. Pat. No. 8,662,369, Specification

### SUMMARY OF INVENTION

### Technical Problem

An object of the present invention is to provide a medical staple with which it is possible to stably cause bending deformation into a predetermined shape by means of a small amount of force.

### Solution to Problem

An aspect of the present invention is a medical staple including a rectangular base portion; and a needle portion that extends from one end of the base portion in a substantially arc shape so as to protrude in a predetermined direction with respect to the base portion, wherein the needle portion has a first bending position that is provided between a base connected at the one end and a distal end, and that yields first when a pressing force in a radially inward direction of a substantially arc shape of the needle portion acts on the distal end, and a second bending position that is provided between the first bending position and the base, and that yields first when the pressing force acts in the vicinity of the base of the first bending position.

### BRIEF DESCRIPTION OF DRAWINGS

FIG. 1A shows a plan view, viewed from a needle-portion side according to an embodiment of the present invention.

FIG. 1B shows a front view showing an overall configuration of a medical staple according to an embodiment of the present invention.

FIG. 2A shows cross-sectional views taken along a first bending position of a needle portion provided in the medical staple in FIGS. 1A and 1B.

FIG. 2B shows cross-sectional views taken along a second bending position of a needle portion provided in the medical staple in FIGS. 1A and 1B.

FIG. 2C shows cross-sectional views taken along a base of a needle portion provided in the medical staple in FIGS. 1A and 1B.

FIG. 3A shows front views of the medical staple in FIG. 1B bent at the first bending position and.

FIG. 3B shows front views of the medical staple in FIG. 1B bent at the second bending position.

FIG. 4A is a diagram showing in the process of suturing biological tissue by using the medical staple in FIG. 1B.

FIG. 4B is a diagram showing in the process of suturing biological tissue by using the medical staple in FIG. 1B.

FIG. 4C is a diagram showing, in the process of suturing biological tissue by using the medical staple in FIG. 1B.

FIG. 4D is a diagram showing, in changes in the amount of force required to cause bending deformation in the medical staple.

FIG. 5A shows a plan view, viewed from a needle-portion side showing a modification of the medical staple in FIG. 1A.

FIG. 5B shows a front view showing a modification of the medical staple in FIG. 1B.

FIG. 5C shows a left side view, showing a modification of the medical staple in FIG. 1A and 1B.

FIG. 6A shows a front view showing another modification of the medical staple in FIG. 1B.

FIG. 6B shows a front view showing a state in which the staple of 6A is bent at the first bending position and the second bending position.

FIG. 7 is a front view showing another modification of the medical staple in FIG. 1B.

FIG. 8 is a front view showing another modification of the medical staple in FIG. 1B.

### DESCRIPTION OF EMBODIMENT

A medical staple 1 according to an embodiment of the present invention will be described below with reference to the drawings.

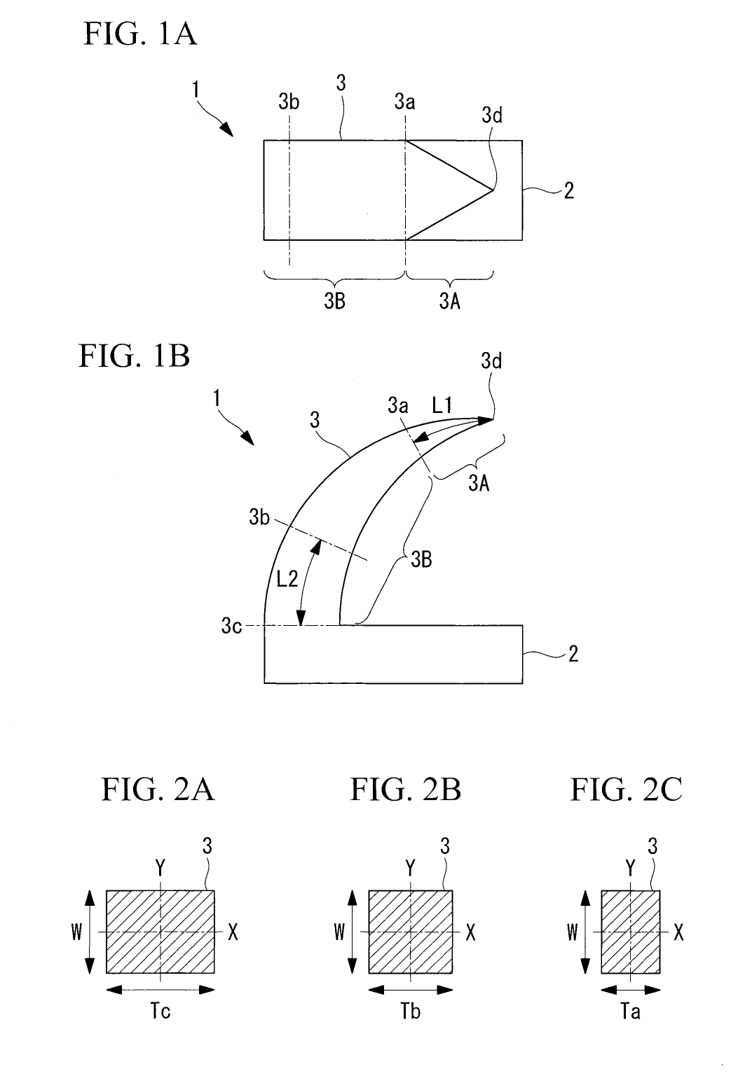

As shown in FIGS. 1A and 1B, the medical staple according to this embodiment is provided with a rectangular base portion 2 and a single needle portion 3 that extends from one end of the base portion 2.

The base portion 2 and the needle portion 3 are formed of a highly biocompatible metal, such as a titanium alloy, stainless steel, or the like.

The needle portion 3 protrudes in a predetermined direction with respect to the base portion 2, and is arc-shaped in which the center angle thereof is substantially 90° or less than 90°. In other words, the needle portion 3 protrudes from one end of the base portion 2 in a longitudinal direction thereof, extends toward the other end of the base portion 2 by protruding in a direction that intersects the longitudinal direction of the base portion 2, and has a distal end 3d as a free end. An outer-circumferential surface of the needle portion 3 that is positioned radially outside is curved at a constant curvature.

In the following, a direction (in FIG. 1B, a direction perpendicular to the plane of the figure) perpendicular to a plane defined by the base portion 2 and the needle portion 3 (in FIG. 1B, the same plane as the plane of the figure) is defined as the width direction of the needle portion 3, and a radial direction of the arc shape of the needle portion 3 is defined as the thickness direction of the needle portion 3.

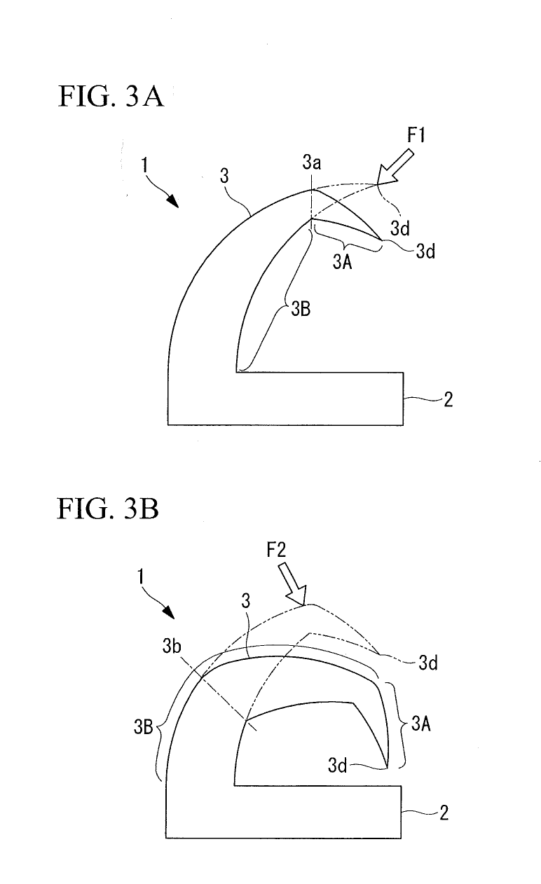

The needle portion 3 is formed of, sequentially from the base portion 2, a curved portion 3B and a distal-end portion 3A that has the sharp distal end 3d. The curved portion 3B has a constant width W, and the distal-end portion 3A has a width that gradually decreases toward the distal end 3d. In addition, the needle portion 3 has thicknesses Tc, Tb, and Ta that gradually decrease from a base 3c, which is connected at the base portion 2, toward the distal end 3d (i.e., Tc>Tb>Ta). Accordingly, as shown in FIGS. 2A, 2B, and 2C, the substantially rectangular cross-sectional area of the needle portion 3 in the thickness direction thereof gradually decreases from the base 3c toward the distal end 3d. FIG. 2A shows the cross section at a first bending position 3a, which is a boundary between the distal-end portion 3A and the curved portion 3B; FIG. 2B shows the cross section at a second bending position 3b, which is an intermediate position in the curved portion 3B; and FIG. 2C shows the cross section at the base 3c. In addition, by forming the distal-end portion 3 A in a shape in which the diameter thereof gradually decreases toward the sharp distal end 3d, the distal-end portion 3A is configured so as to easily pierce or pass through biological tissue.

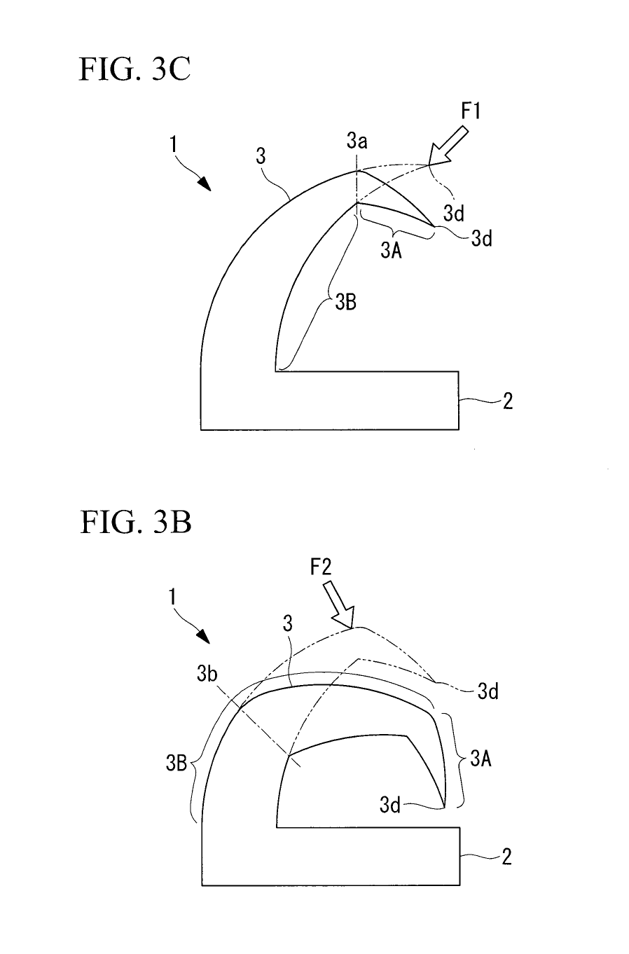

As shown in FIG. 3A, the needle portion 3 is designed so as to yield first at the first bending position 3a and to be bent about an axis in the width direction when a pressing force F1 in a radially inward direction of the arc shape of the needle portion 3 acts on the distal end 3d, thus increasing the pressing force F1. Furthermore, as shown in FIG. 3B, the needle portion 3 is designed so as to yield first at the second bending position 3b and to be bent about an axis in the width direction when a pressing force F2 in a radially inward direction of the arc shape of the needle portion 3 acts in the vicinity of the base of the first bending position 3a, thus increasing the pressing force F2.

Here, bending-deformation characteristics of the needle portion 3 will be described.

The bending deformation that can occur in the needle portion 3 when a pressing force acts on the needle portion 3 can be divided into bending deformation about an axis in the thickness direction (X-axis) and bending deformation about an axis in the width direction (Y-axis), and the difficulty involved in causing the bending deformation about the respective axes depends on the cross-sectional shapes. Specifically, a cross-sectional second-order moment Ix about the X-axis and a cross-sectional second-order moment Iy about the Y-axis are defined by the expressions below:

Ix=(1/12)*thickness*(width)̂3, and

Iy=(1/12)*(thickness)̂3*width.

When the pressing force F1 or F2 acts on the distal end 3d of the needle portion 3 or in the vicinity of the base 3c of the first bending position 3a thereof, the magnitudes of the bending moments about the Y-axis in the width direction that act at the respective positions of the needle portion 3 depend on the distances between these positions and the positions at which the pressing forces F1 and F2 are acting and the angles formed between the thickness directions at these positions and the directions in which the pressing forces F1 and F2 are acting.

On the basis of such a relationship between the bending moment and the cross-sectional second-order moment, the shape of the needle portion 3 is designed so that the first bending position 3a yields first to the bending moment when the pressing force Fl acts on the distal end 3d. Similarly, the shape of the needle portion 3 is designed so that the second bending position 3b between the base 3c and the first bending position 3a yields first to the bending moment when the pressing force F2 acts in the vicinity of the base of the first bending position 3a.

As shown in FIG. 2B, the thickness Tb and the width W of the cross-sectional shape are the same at the second bending position 3b, and, as shown in FIG. 2C, the width W is greater than the thickness at a position that is closer to the distal end 3d than the second bending position 3b is. In other words, at the first bending position 3a and the second bending position 3b, the cross-sectional second-order moment about the Y-axis is equal to or less than the cross-sectional second-order moment about the axis in the other direction, and thus, in the portion between the second bending position 3b and the distal end 3d, in which the cross-sectional area is relatively small in the needle portion 3, it is particularly difficult to cause bending deformation about the X-axis, as compared with that about the Y-axis. By doing so, it is easier to control the bending directions at the first bending position 3a and the second bending position 3b in the direction parallel to the plane, and thus, the bent shape of the needle portion 3 is made more stable. A length L1 between the distal end 3d and the first bending position 3a in a circumferential direction is smaller than a length L2 between the second bending position 3b and the base in a circumferential direction.

Next, the operation of the thus-configured medical staple 1 will be described.

The medical staples 1 according to this embodiment are used as needles for a medical stapler provided with a cartridge 4 and an anvil 5 that can be opened and closed. The cartridge 4 and the anvil 5 have inner surfaces 4a and 5a that face each other and are configured so that biological tissue can be gripped between the inner surfaces 4a and 5a in a state in which the cartridge 4 and the anvil 5 are closed.

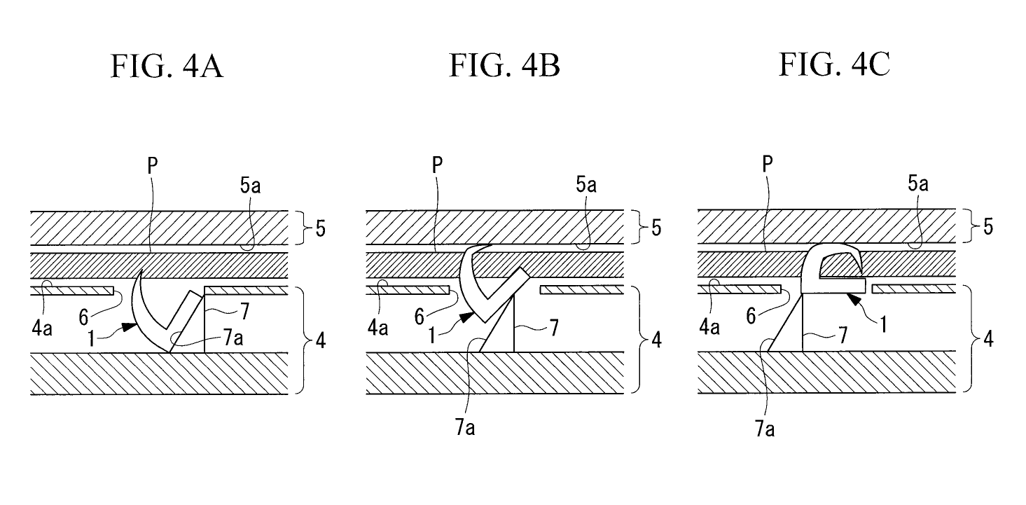

Numerous slots 6 are provided in the inner surface 4a of the cartridge 4. The medical staples 1 are accommodated in the slots 6 so as to be protrudable from and retractable into the slots 6 in a state in which the distal ends 3d face the inner surface 5a of the anvil 5. The cartridge 4 is provided, in a movable manner, with cams 7 for pushing out the medical staples 1 from the slots 6. When the base portions 2 of the medical staples 1 are pushed by inclined faces 7a provided in the cams 7 sequentially from one ends thereof to base ends thereof, the medical staples 1 are pushed out from the slots 6 while being rotated along the arc shapes of the needle portions 3, as shown in FIG. 4A.

The needle portions 3 that are pushed out from the slots 6 while being rotated pass through biological tissue P from the cartridge 4 side in the thickness direction, and, subsequently, the distal ends 3d come into contact with the inner surface 5a of the anvil 5. Then, the pressing force F1 from the inner surface 5a of the anvil 5 acts on the distal ends 3d, and thus, the first bending positions 3a begin to bend, as shown in FIG. 4B. With an increase in the amounts by which the needle portions 3 are pushed out from the slots 6, the positions at which the needle portions 3 come into contact with the inner surface 5a of the anvil 5 move along the outer-circumferential surfaces of the needle portions 3 from the distal ends 3d toward the first bending positions 3a, and, during this movement, the bend angles of the first bending positions 3a are gradually increased.

When the positions at which the needle portions 3 come into contact with the inner surface 5a of the anvil 5 pass the first bending positions 3a, bending deformation of the first bending positions 3a is completed, and, next, the pressing force F2 from the inner surface 5a acts in the vicinity of the bases of the first bending positions 3a, and thus, the second bending positions 3b begin to bend. With an increase in the amounts by which the needle portions 3 are pushed out from the slots 6, the positions at which the needle portions 3 come into contact with the inner surface 5a of the anvil 5 move along the outer-circumferential surfaces of the needle portions 3 from the vicinity of the bases of the first bending positions 3a toward the bases 3c, and, during this movement, the bend angles of the second bending positions 3b are gradually increased. When the second bending positions 3b begin to bend, the distal ends 3d move toward the cartridge 4, and thus, the distal-end portions 3A pierce the biological tissue P again from the anvil 5 side, as shown in FIG. 4C. By doing so, it is possible to stably suture the biological tissue at two positions.

Because the lengths L1 between the distal ends 3d and the first bending positions 3a of the needle portions 3 are smaller than the lengths L2 between the second bending positions 3b and the bases 3c, the distal ends 3d do not come into contact with the cartridge 4 even if the bend angles of the second bending positions 3b exceed 90°. Therefore, it is possible to bend the second bending positions 3b until reaching a satisfactory angle so that the biological tissue P is tightly held between the base portions 2 and portions between the first bending positions 3a and the second bending positions 3b of the needle portions 3, and thus, it is possible to more reliably suture the biological tissue P.

When the positions at which the needle portions 3 come into contact with the inner surface 5a of the anvil 5 pass the second bending positions 3b, bending deformation of the second bending positions 3b is completed, and, next, the pressing force from the inner surface 5a acts between the second bending positions 3b and the bases 3c, and thus, portions between the second bending positions 3b and the bases 3c are bent.

As has been described above, with this embodiment, deformation is caused by means of contact between the outer-circumferential surface of the arc shape of the needle portion 3 and the inner surface 5a of the anvil 5, it is possible to cause deformation by means of an amount of force that is smaller than the case of the staples in the related art. In particular, because the outer-circumferential surface of the needle portion 3 has the constant curvature, with an increase in the amount by which the needle portion 3 is pushed out from the cartridge 4, the outer-circumferential surface of the needle portion 3 that comes into contact with the inner surface 5a of the anvil 5 moves while smoothly being rotated with respect to the inner surface 5a. By doing so, there is an advantage in that it is possible to further decrease the amount of force required to cause bending deformation in the needle portion 3.

The first bending position 3a and the second bending position 3b, which preferentially yield to the pressing forces F1 and F2 applied thereto from the anvil 5 in the process of causing bending deformation by means of the anvil 5, are set in the needle portion 3. After the first bending position 3a is bent by yielding to the pressing force F1, because the bending rigidity of the first bending position 3a is considerably decreased, the pressing force that is subsequently applied to the needle portion 3 from the anvil 5 is consumed to cause additional bending deformation in the first bending position 3a, and thus, deformation of portions other than the first bending position 3a is prevented. Similarly, after the second bending position 3b is bent by yielding to the pressing force F2, because the pressing force that is applied to the needle portion 3 from the anvil 5 is consumed to cause additional bending deformation in the second bending position 3b, deformation of portions other than the second bending position 3b is prevented. As has been described above, because the bending positions of the needle portion 3 are restricted to the first bending position 3a and the second bending position 3b, there is an advantage in that it is possible to form the needle portion 3 into a predetermined bent shape by means of an even smaller amount of force.

As shown in FIGS. 2A, 2B, and 2C, because the cross-sectional area of the needle portion 3 gradually increases from the distal end 3d toward the base 3c, it becomes increasingly difficult to cause bending deformation in the needle portion 3 in portions thereof that are closer to the base. In particular, the cross-sectional second-order moment about the Y-axis strongly depends on the thickness. Therefore, because the thickness gradually increases from the distal end 3d toward the base 3c, a portion that is closer to the base 3c than the first bending position 3a is stably endures the bending moment generated by the pressing force Fl when the pressing force F1 acts on the distal end 3d. Similarly, a portion that is closer to the base 3c than the second bending position 3b is stably endures the bending moment generated by the pressing force F2 when the pressing force F2 acts in the vicinity of the base of the first bending position 3a. By doing so, it is possible to reliably bend the needle portion 3 only at the first bending position 3a and the second bending position 3b, and thus, there is an advantage in that it is possible to further stabilize the bent shape of the needle portion 3.

The needle portion 3 possesses characteristics that make it difficult to cause bending deformation about the X-axis. Therefore, for example, even if bending moment about the X-axis acts on the needle portion 3 in such a way that the needle portion 3 is pushed out from the slot 6 in a slightly slanted manner instead of perpendicular to the inner surface 5a of the anvil 5 or the like, the needle portion 3 stably endures the bending moment about the X-axis, and thus, the bending deformation thereof is selectively caused only about the Y-axis. By doing so, there is an advantage in that it is possible to further stabilize the bent shape of the needle portion 3.

At the first bending position 3a and the second bending position 3b, it is possible to cause bending deformation by means of smaller amounts of forces as compared with other portions of the needle portion 3. Thus, after the first bending position 3a and the second bending position 3b have yielded once, it is also possible to make the amounts of forces required to cause additional bending deformation at the first bending position 3a and the second bending position 3b small, as shown in FIG. 4D. Therefore, there is an advantage in that it is possible to cause bending deformation in the needle portion 3 by means of a small amount of force throughout the bending deformation process.

The width of the needle portion 3 is constant from the base 3c to the first bending position 3a, the thickness of the needle portion 3 is smaller at the second bending position 3b than it is at the base 3c, and the thickness is smaller at the first bending position 3a than it is at the second bending position 3b. By forming the needle portion 3 in such a shape, processing and manufacturing of the needle portion 3 are facilitated. Because the widths by which the curved portion 3B and the inner surface 5a of the anvil 5 come into contact with each other are constant, there is an advantage in that the shape of the anvil pocket is simplified and that it is possible to stably deform the needle portion 3.

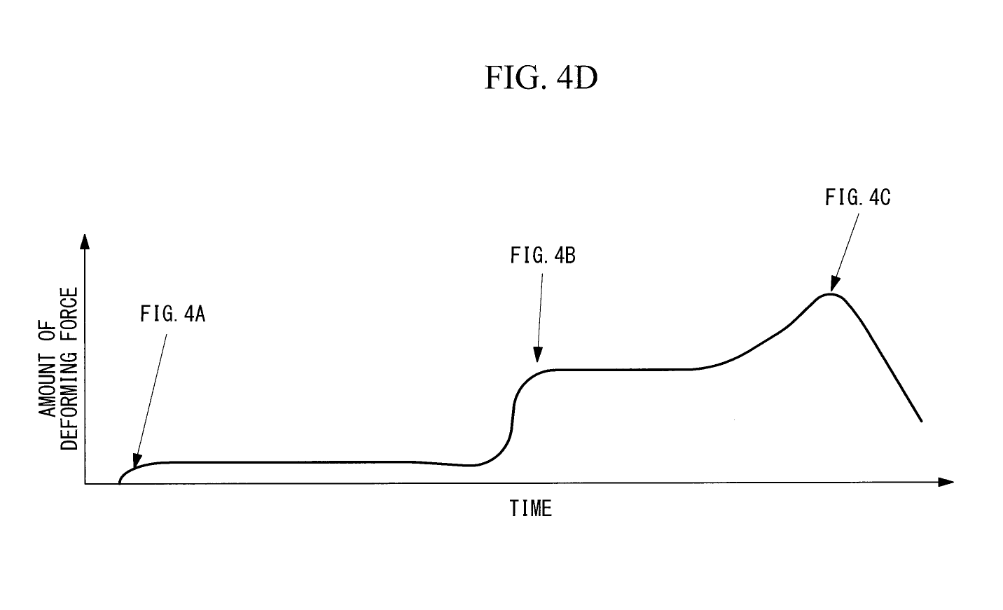

In this embodiment, although the medical staple 1 has been assumed to have a single needle portion 3, alternatively, the medical staple 1 may be provided with two or more needle portions 3, as shown in FIGS. 5A, 5B, and 5C.

The medical staple 1 shown in FIGS. 5A, 5B, and 5C is provided with two needle portions 3. The needle portions 3 protrude from two positions at one end of the base portion 2 with a space therebetween in the width direction, and extend so as to be parallel to each other.

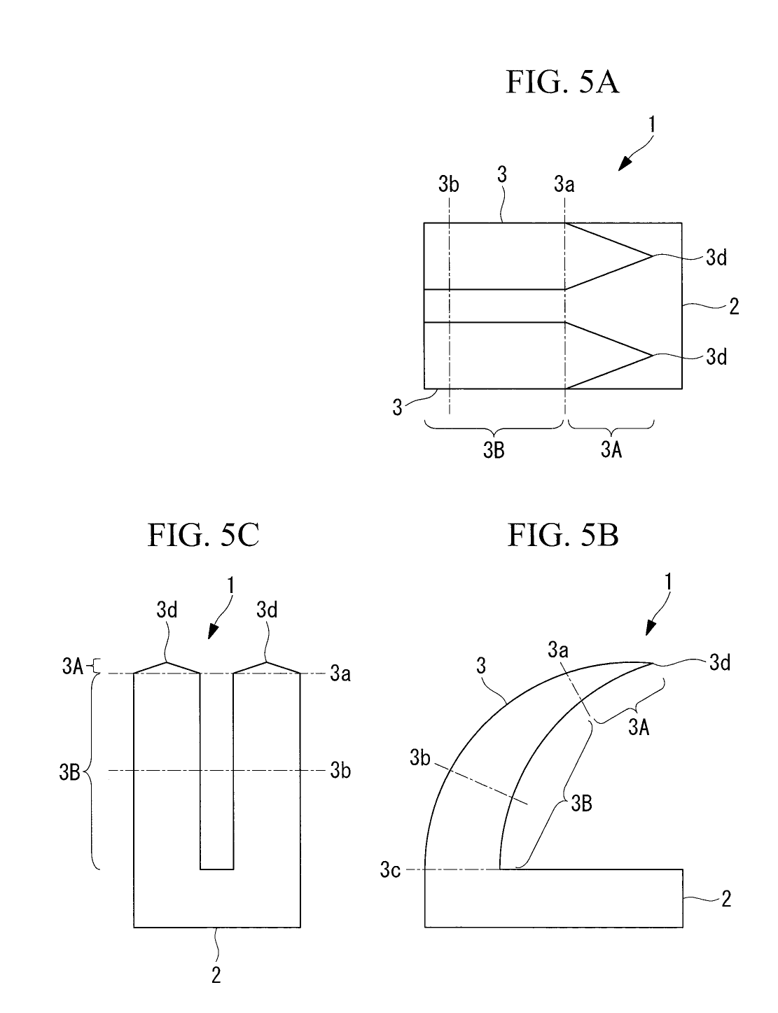

As shown in FIG. 6A, the two needle portions 3 may be provided at both ends of the base portion 2. In this case, the two needle portions 3 protrude from both ends of the base portion 2 in opposing directions from each other in the longitudinal direction of the base portion 2, and are curved in the same direction with respect to the base portion 2. As shown in FIG. 6B, it is possible to cause bending deformation in the needle portion 3 having such a shape, by using, for example, an anvil in which the inner surface 5a thereof has a shape such as that shown in FIG. 6A.

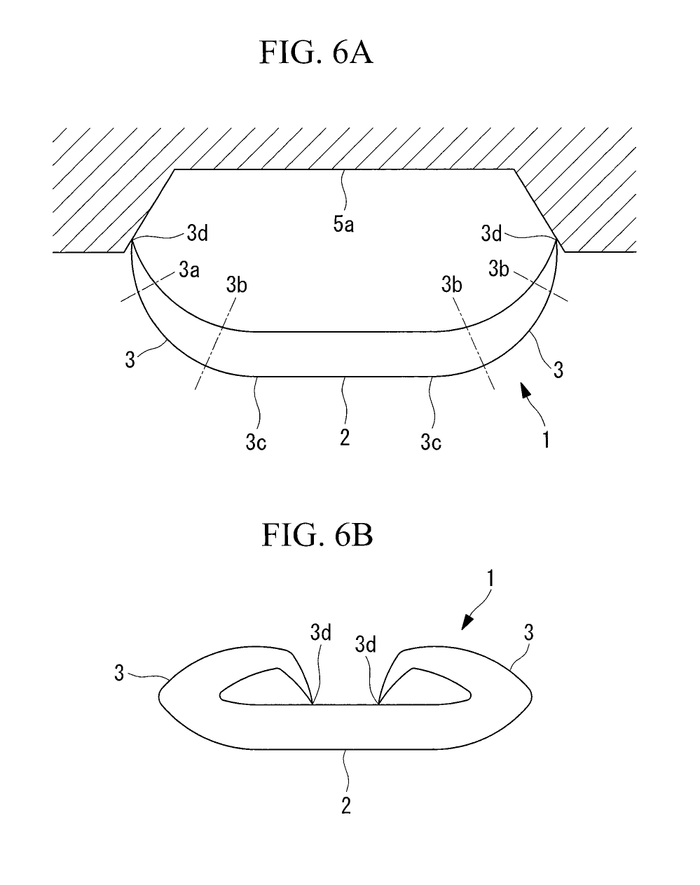

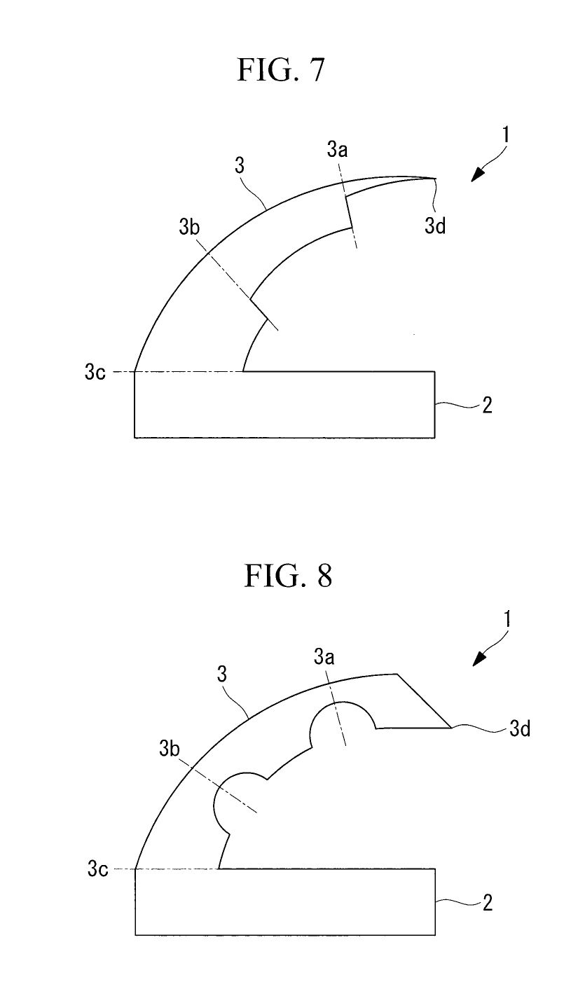

In this embodiment, although the needle portion 3 is assumed to have a shape in which the width and the thickness thereof continuously change from the distal end 3d to the base 3c, alternatively, the needle portion 3 may have a shape in which the width and the thickness thereof change in a step-wise manner, as shown in FIGS. 7 and 8.

Doing so facilitates designing the cross-sectional second-order moments Ix and Iy for the respective positions of the needle portion 3, and thus, it is possible to easily design a shape of the needle portion 3 such that the first bending position 3a and the second bending position 3b first yield to the pressing forces F1 and F2, respectively.

### REFERENCE SIGNS LIST

1 medical staple
2 base portion
3 needle portion
3A distal-end portion
3B curved portion
3a first bending position
3b second bending position
3c base
3d distal end

1. A medical staple comprising:
a rectangular base portion; and
a needle portion that extends from one end of the base portion in a substantially arc shape so as to protrude in a predetermined direction with respect to the base portion,
wherein the needle portion has
a first bending position that is provided between a base connected at the one end and a distal end, and that yields first when a pressing force in a radially inward direction of a substantially arc shape of the needle portion acts on the distal end, and
a second bending position that is provided between the first bending position and the base, and that yields first when the pressing force acts in the vicinity of the base of the first bending position.

2. A medical staple according to claim 1, wherein, at the first bending position and the second bending position of the needle portion, a cross-sectional second-order moment about an axis in a direction perpendicular to a plane defined by the base portion and the needle portion is equal to or less than a cross-sectional second-order moment about an axis in another direction.

3. A medical staple according to claim 1, wherein an outer-circumferential surface of the needle portion has a constant curvature.

4. A medical staple according to claim 1, wherein a cross-sectional area of the needle portion is greater at the second bending position than at the first bending position, and the cross-sectional area is greater at the base than at the second bending position.

5. A medical staple according to claim 4,
wherein a width of the needle portion is constant between the first bending position and the base,
a thickness of the needle portion is smaller at the second bending position than at the base, and the thickness is smaller at the first bending position than at the second bending position.

6. A medical staple according to claim 1,
wherein the needle portion has a distal-end portion formed of a portion between the distal end and the first bending position,
the cross-sectional area of the needle portion is smaller at the distal-end portion than at a portion between the first bending position and the base, and
a size of the needle portion is smaller in the distal-end portion than in a portion between the second bending position and the base.
